(19) United States
(12) Patent Application Publication
(10) Pub. No.: US 2026/0069817 A1
(43) Pub. Date: Mar. 12, 2026

(54) METHOD FOR OPERATING A RESPIRATORY AIR HUMIDIFIER

(71) Applicant: Loewenstein Medical Technology S.A., Luxembourg (LU)

(72) Inventors: Jochen FURTHMUELLER, Rheinstetten (DE); Matthias SCHWAIBOLD, Karlsruhe (DE)

(21) Appl. No.: 19/279,064

(22) Filed: Jul. 24, 2025

(30) Foreign Application Priority Data
Sep. 12, 2024 (DE) 10 2024 126 301.6

(51) Int. Cl. A61M 16/16; A61M 16/00; A61M 16/10

(52) U.S. Cl. CPC A61M 16/16; A61M 16/0003; A61M 16/024; A61M 16/109; A61M 2205/3334; A61M 2205/3372; A61M 2205/36

(57) ABSTRACT

A respiratory air humidifier comprises a respiratory air inlet, a respiratory air outlet and an electrical heating element. The humidifier can be switched between a humidification mode and a preparatory mode to prepare the humidifier for operation in the humidification mode and is formed in order, in the humidification mode, to humidify respiratory air supplied at the respiratory air inlet, the respiratory air being thermally regulated by the heating element, and to provide it in the humidified state at the respiratory air outlet. A method for operating the humidifier comprises: when the humidifier is switched on or off, switching the humidifier to the preparatory mode and operating the humidifier in the preparatory mode in order to enable dissipation of excess heat, which is due to operation of the humidifier in the humidification mode prior to switching on or off, from the interior of the humidifier.

### CROSS-REFERENCE TO RELATED APPLICATIONS

The present application claims priority under 35 U.S.C. § 119 of German Patent Application No. 10 2024 126 301.6, filed Sep. 12, 2024, the entire disclosure of which is expressly incorporated by reference herein.

### BACKGROUND OF THE INVENTION

### 1. Field of the Invention

The invention relates to a method for operating a respiratory air humidifier. The invention furthermore relates to a control unit, to a computer program and to a computer-readable medium for carrying out the method, as well as to a respiratory air humidifier.

### 2. Discussion of Background Information

Artificially ventilated patients may sometimes suffer from excessively dry airways. In such cases, the respiratory air to be inhaled may be additionally humidified with the aid of a respiratory air humidifier, while being brought to a suitable temperature by means of an electrical heating element. Drying of the mucous membranes may therefore be avoided, or at least alleviated. If the ventilation therapy is briefly interrupted, it may be the case that the respiratory air in the interior of the respiratory air humidifier continues to be passively humidified and/or heated because the heating element is still warm, despite being switched off. This may lead to the inhaled respiratory air feeling unpleasantly warm and/or unpleasantly humid during the first few breaths after the ventilation therapy is resumed.

In view of the foregoing, it would be advantageous to have available a method for operating a respiratory air humidifier, with which it is possible to prevent the inhaled respiratory air from feeling unpleasantly warm and/or unpleasantly humid when the respiratory air humidifier is switched on after a brief operating interruption. It further would be advantageous to have available a control unit, a computer program and a computer-readable medium for carrying out such a method, as well as a corresponding respiratory air humidifier.

### SUMMARY OF THE INVENTION

In a first aspect the invention provides a method for operating a respiratory air humidifier, in particular for a ventilator. The respiratory air humidifier comprises a respiratory air inlet, a respiratory air outlet and an electrical heating element. The respiratory air humidifier can be switched between a humidification mode and a preparatory mode to prepare the respiratory air humidifier for operation in the humidification mode and is formed in order, in the humidification mode, to humidify respiratory air which is supplied at the respiratory air inlet, the respiratory air being thermally regulated by means of the heating element (i.e. brought to a temperature that feels comfortable when inhaling by controlling the heating element), and to provide it in the humidified state at the respiratory air outlet. The method comprises: when the respiratory air humidifier is switched on or off, switching the respiratory air humidifier to the preparatory mode and operating the respiratory air humidifier in the preparatory mode in order to enable (or accelerate) dissipation of excess heat, which is due to operation of the respiratory air humidifier in the humidification mode prior to switching on or off, from the interior of the respiratory air humidifier. During operation of the respiratory air humidifier in the preparatory mode, the heating element is deactivated.

In other words, each time the respiratory air humidifier is switched on or off, or more accurately each time it is detected that the respiratory air humidifier is (or is intended to be) switched on or off, the respiratory air humidifier can be operated automatically for a particular time in the preparatory mode in order to enable sufficient passive and/or active cooling of the heating element and/or of the air in the interior of the respiratory air humidifier before the reactivation of the humidification mode, for example before the resumption of a briefly (e.g. for less than about 10 minutes, less than about 5 minutes or less than about 3 minutes) interrupted ventilation therapy. Alternatively, the switching to the preparatory mode may take place temperature-dependently. In this case, it is possible that the respiratory air humidifier is only switched to the preparatory mode when it is switched on or off when a particular temperature has been reached in the interior of the respiratory air humidifier (see below).

The dissipation of the excess heat may take place passively and/or actively, for example by correspondingly controlling one or more electropneumatic actuators.

It is possible that the respiratory air humidifier is operated in the preparatory mode for a rigidly predefined operating duration (e.g. from about 10 seconds to about 5 minutes).

Alternatively, the operating duration may be predefined as a function of a current (measured and/or estimated) temperature in the interior of the respiratory air humidifier.

The operation of the respiratory air humidifier in the preparatory mode may also be restricted only to preventing the respiratory air humidifier from being switched to the humidification mode and/or to preventing the heating element from being activated.

The humidification mode—in contrast to the preparatory mode—may be a main type of operation of the respiratory air humidifier. The respiratory air humidifier may be operated differently in the preparatory mode than in the humidification mode. In principle, the respiratory air humidifier may be operated in the preparatory mode so that a likelihood that the respiratory air provided at the respiratory air outlet feels unpleasantly warm, and/or unpleasantly humid, when it is inhaled shortly after the respiratory air humidifier is switched on is reduced significantly in comparison to operation of the respiratory air humidifier in the humidification mode.

It is possible that the heating element is activated each time the respiratory air humidifier is switched on and/or each time the respiratory air humidifier is switched to the humidification mode, and/or is deactivated each time the respiratory air humidifier is switched off and/or each time the respiratory air humidifier is switched to the preparatory mode. In other words, the heating element may be activated during the (entire) operation of the respiratory air humidifier in the humidification mode, and/or deactivated during the (entire) operation of the respiratory air humidifier in the preparatory mode. The heating element may be switched on continuously in the activated state. The heating power of the heating element may in this case be varied suitably in order to thermally regulate the respiratory air, for example by varying an electric current flowing through the heating element and/or an electric voltage applied to the heating element (e.g. by pulse-width modulation). In addition or alternatively, the (average) heating power of the heating element in the activated state may be controlled by repeatedly switching the heating element alternately on and off. On the other hand, the heating element in the deactivated state may be continuously switched off, i.e. electrically disconnected from a power supply, so that the respiratory air is not—or at least not deliberately or actively—heated by means of the heating element.

If the respiratory air humidifier is being operated in the humidification mode, it is possible that the respiratory air humidifier can be switched to the preparatory mode again only if the respiratory air humidifier is switched off beforehand. In other words, once the respiratory air humidifier has been operated in the preparatory mode, it may be possible to switch the respiratory air humidifier to the preparatory mode once more only after the respiratory air humidifier is switched on, or off, once more. For example, the operation of the respiratory air humidifier in the humidification mode can be interrupted only by switching the respiratory air humidifier off—and not by switching the respiratory air humidifier to the preparatory mode.

The respiratory air humidifier may be powered down when it is switched off. In other words, the respiratory air humidifier may be electrically disconnected from a power supply, for example an electrical socket and/or a battery. Alternatively, the respiratory air humidifier may be switched to power saving operation when it is switched off. The (switched off) respiratory air humidifier may continue to be supplied with power (for example from an additional battery) in power saving operation, but while consuming much less energy than in the on state. In particular, the heating element may be deactivated in power saving operation. Accordingly, it is possible that the power saving operation is ended again when the respiratory air humidifier is switched on, for example when the respiratory air humidifier is switched to the humidification mode (instead of to the preparatory mode) when switching on. Alternatively, the power saving operation may be maintained when switching on if the respiratory air humidifier is switched to the preparatory mode when switching on. In other words, the (switched on) respiratory air humidifier may consume much less energy in the preparatory mode than in the humidification mode.

“Respiratory air” may be understood as a single respiratory gas or a mixture of a plurality of respiratory gases for the ventilation of a patient. “Respiratory gas” may for example be understood as carbon dioxide, oxygen, nitrogen, water vapor or anesthetic gas.

Humidification of the respiratory air may be understood as deliberate mixing of the respiratory air with water vapor and/or water droplets in combination with deliberate heating of the respiratory air.

The method may, for example, be computer-implemented.

In a second aspect the invention provides a control unit which is configured to carry out the method described above and below.

The control unit may comprise data processing elements. The data processing elements may be implemented as hardware and/or software, and/or may comprise a processor. The processor may be configured to carry out the (computer-implemented) method. In addition to the processor, the control unit may comprise at least one of the following data processing elements: a memory, a bus system for data communication between the memory and the processor, a data communication interface for wireless and/or wired data communication with peripheral devices. Alternatively, the control unit may be implemented exclusively as hardware, for example in the form of an ASIC module or FPGA module.

It should be pointed out that features of the method described above and below may also be features of the control unit (and vice versa).

In a third aspect the invention provides a respiratory air humidifier, in particular for a ventilator. The respiratory air humidifier comprises a respiratory air inlet, a respiratory air outlet, an electrical heating element and a control unit as described above and below. The respiratory air humidifier can be switched between a humidification mode and a preparatory mode to prepare the respiratory air humidifier for operation in the humidification mode and is formed in order, in the humidification mode, to humidify respiratory air which is supplied at the respiratory air inlet, the respiratory air being thermally regulated by means of the heating element, and to provide it in the humidified state at the respiratory air outlet.

The respiratory air inlet may, for example, be attachable to a ventilator for the invasive and/or noninvasive ventilation of a patient, in order to enable humidification of the respiratory air provided by the ventilator before the respiratory air is inhaled by the patient. A “ventilator” may also be understood as an anesthesia device or a cough assist device for assisting a patient when coughing, also known as an insufflator-exsufflator.

The respiratory air outlet may, for example, be attachable to the patient's respiratory system via one or more ventilation hoses and/or via a patient interface (for example a mask, a nasal cannula and/or tubing) so that ventilation of the patient with the humidified respiratory air is possible.

The respiratory air humidifier may be formed in order to humidify the respiratory air by the respiratory air on the one hand being mixed with water vapor and/or water droplets, and on the other hand being thermally regulated by direct and/or indirect contact with the heating element. For example, the respiratory air humidifier may be formed in order to generate water vapor for humidifying the respiratory air by heating water by means of the heating element and/or water droplets for humidifying the respiratory air, in particular an aerosol, by atomizing water by means of an atomizer, for example a piezoelectric ultrasonic atomizer.

The control unit may, for example, be configured to control the heating element and/or one or more electropneumatic actuators during operation of the respiratory air humidifier in the humidification mode so that an actual temperature of the respiratory air provided at the respiratory air outlet approximates a desired comfort temperature at which the respiratory air feels pleasantly warm when inhaling. The comfort temperature may, for example, lie between 30° C. and 38° C.

Correspondingly, the control unit may be configured to control the heating element and/or one or more electropneumatic actuators during operation of the respiratory air humidifier in the humidification mode so that an actual relative air humidity of the respiratory air provided at the respiratory air outlet, expressed in terms of a respective ambient temperature, approximates a desired relative air humidity at which the respiratory air feels pleasantly humid when inhaling.

The term “electrical heating element” may generally be understood as an electrically controllable heating element, in particular a heating resistor. The heating element may for example comprise a heating rod, a heating filament and/or a heating plate. The heating element may be formed in order to thermally regulate the respiratory air directly and/or indirectly, for example by heating a liquid with which the respiratory air comes in contact when flowing through the respiratory air humidifier.

The respiratory air humidifier may be embodied as an independent device and/or as a component of a medical device, in particular of a ventilator.

Further aspects of the invention relate to a computer program and to a computer-readable medium, on which the computer program is stored.

The computer program comprises instructions which cause the control unit described above and below, for example a processor of the control unit, to carry out the method described above and below when the computer program is run by the control unit.

The computer-readable medium may be a volatile or nonvolatile data memory. For example, the computer-readable medium may be a hard disk drive, a USB storage device (universal serial bus), a RAM (random-access memory), a ROM (read-only memory), an EPROM (erasable programmable read-only memory), an EEPROM (electrically erasable programmable read-only memory), a flash memory or a combination of at least two of these examples. The computer-readable medium may also be a data communication network that makes it possible to download program code (for example via the Internet), or a cloud.

It should be pointed out that features of the method described above and below may also be features of the computer program and/or of the computer-readable medium (and vice versa).

Various embodiments of the invention are described below. These embodiments are not to be understood as a restriction of the scope of the invention.

According to one embodiment, the method may further comprise, when the respiratory air humidifier is switched on: switching the respiratory air humidifier to the humidification mode subsequent to the operation of the respiratory air humidifier in the preparatory mode, and operating the respiratory air humidifier in the humidification mode, for example until the respiratory air humidifier is switched off again. In other words, the respiratory air humidifier may be switched automatically to the humidification mode subsequent to the preparatory mode. The respiratory air humidifier may, for example, be operated in the preparatory mode until it is detected (for example by a control unit of the respiratory air humidifier) that a particular switching criterion for switching the respiratory air humidifier to the humidification mode is fulfilled. Such a switching criterion may, for example, be the elapse of a predefined operating duration of the respiratory air humidifier in the preparatory mode starting from an instant at which the respiratory air humidifier is switched on or off, and/or the reaching of a predefined temperature in the interior of the respiratory air humidifier. It is possible that the respiratory air humidifier is switched to the preparatory mode only one single time each time it is switched on and, after it has been switched to the humidification mode, it can be switched to the preparatory mode again only when it is switched off, or off and on again.

According to one embodiment, the method may further comprise, when the respiratory air humidifier is switched on or off: receiving a temperature value which indicates a current temperature of the respiratory air in the interior of the respiratory air humidifier; comparing the temperature value with a threshold value, which indicates a temperature threshold at which the respiratory air just still feels comfortable when inhaling; if the temperature value exceeds the threshold value: switching the respiratory air humidifier to the preparatory mode and operating the respiratory air humidifier in the preparatory mode. In other words, the switching of the respiratory air humidifier to the preparatory mode may take place with temperature-based control. This has the advantage that the respiratory air humidifier is operated in the preparatory mode only when this is actually necessary. The temperature value may be a measurement value, i.e. a value determined by means of a temperature sensor, and/or an estimated value.

In addition or alternatively, the respiratory air humidifier may—if the temperature value does not exceed the threshold value—be prevented from being switched to the preparatory mode.

According to one embodiment, the method may further comprise, when the respiratory air humidifier is switched on: if the temperature value does not exceed the threshold value: switching the respiratory air humidifier to the humidification mode and operating the respiratory air humidifier in the humidification mode, for example until the respiratory air humidifier is switched off again. In other words, the respiratory air humidifier may be switched directly to the humidification mode when switching on, on condition that the respiratory air in the interior of the respiratory air humidifier has cooled sufficiently since the last operation in the humidification mode. An unnecessary activation of the preparatory mode may therefore be avoided.

According to one embodiment, the temperature value may be received in a plurality of successive time intervals and compared with the threshold value in each time interval. The respiratory air humidifier may in this case be operated in the preparatory mode until the temperature value no longer exceeds the threshold value in one of the time intervals. This enables automatic, temperature-dependent ending of the preparatory mode, for example combined with automatic switching of the respiratory air humidifier from the preparatory mode to the humidification mode. Conversely, automatic switching of the respiratory air humidifier from the humidification mode to the preparatory mode—as mentioned above—may for example take place in response to the respiratory air humidifier being switched off, or switched off and on again.

According to one embodiment, the method may further comprise: calculating the temperature value by using a mathematical model of the respiratory air humidifier. At least one input value may in this case be received and entered into the mathematical model. The mathematical model may be configured to convert the at least one input value into the temperature value and to output the temperature value as an output value. The at least one input value may, for example, indicate at least one of the following variables:
a (current) heating power of the heating element;
a (current) filling level of a container for storing a liquid to humidify the respiratory air, for example water or a liquid containing water;
a (for example average) duration of at least one heating phase, in which the heating element was activated, prior to switching on or off;
a (for example average) duration of at least one cooling phase, in which the heating element was deactivated, prior to switching on or off;
a (current) ambient temperature in the vicinity of the respiratory air humidifier;
a (current) relative air humidity expressed in terms of a (current) ambient temperature in the vicinity of the respiratory air humidifier;
a (current) ambient pressure in the vicinity of the respiratory air humidifier.

An “input value” may be understood as a measured and/or estimated value that quantifies the respective variable(s). The at least one input value may, for example, be received and entered into the mathematical model in a plurality of successive time intervals. The mathematical model may in each time interval output the temperature value as an output value associated with the input value or the input values of the respective time interval.

This embodiment makes it possible to determine the temperature value with sufficient accuracy even without the assistance of a separate temperature sensor.

According to one embodiment, the operation of the respiratory air humidifier in the preparatory mode may comprise: generating a control signal for controlling at least one electropneumatic actuator in order to generate an air flow for dissipating the excess heat through the respiratory air inlet and/or through the respiratory air outlet and/or through at least one vent opening of the respiratory air humidifier, which is separate from the respiratory air inlet and from the respiratory air outlet.

The vent opening may, for example, be formed in a housing of the respiratory air humidifier and/or in a housing of a ventilator. The term “electropneumatic actuator” may be understood above and below generally as an electrically controllable actuator for controlling an air flow. For example, the electropneumatic actuator may be a fan and/or an electrically controllable valve, for example a solenoid valve. With the aid of such active thermal dissipation, the cooling of the respiratory air humidifier may be accelerated significantly in comparison to an embodiment with purely passive thermal dissipation.

According to one embodiment, the operation of the respiratory air humidifier in the preparatory mode may comprise: generating a control signal for controlling at least one electropneumatic actuator in order to prevent the respiratory air from being provided at the respiratory air outlet. This way, it is possible to prevent the patient from inhaling respiratory air that is possibly too warm and/or too humid.

According to one embodiment, the operation of the respiratory air humidifier in the preparatory mode may comprise: generating a control signal for controlling at least one electropneumatic actuator in order to provide the respiratory air at the respiratory air outlet with a (significantly) lower pressure and/or a (significantly) lower flow rate and/or a different flow direction than during operation of the respiratory air humidifier in the humidification mode. Because of the lower pressure and/or the lower flow rate, the respiratory air can be prevented from feeling unpleasantly warm and/or unpleasantly humid when inhaling, even if it has not yet cooled sufficiently. The flow rate, or the pressure, in the preparatory mode may for example deviate from the flow rate, or pressure, in the humidification mode by at least about 5%, at least about 10%, at least about 20% or at least about 50%. The flow direction in the preparatory mode may be opposite to the flow direction in the humidification mode. In other words, it is possible that the respiratory air is drawn in at the respiratory air outlet in the preparatory mode instead of—as in the humidification mode—being blown out. In this way, it is possible to prevent the patient from inhaling respiratory air that is possibly too warm and/or too humid.

According to one embodiment, the operation of the respiratory air humidifier in the preparatory mode may comprise: generating a control signal for controlling at least one electropneumatic actuator so that a flow rate of the respiratory air provided at the respiratory air outlet follows a partially or continuously rising flow rate profile and/or a pressure of the respiratory air provided at the respiratory air outlet follows a partially or continuously rising pressure profile. In other words, the flow rate or the pressure, or the flow rate and the pressure, may be increased with a delay in the preparatory mode, for example in stages. Such an (at least partially) rising profile allows the patient to take off a mask or nasal cannula attached to the respiratory air outlet promptly if the respiratory air from the mask or nasal cannula feels unpleasantly warm and/or unpleasantly humid when inhaling. The electropneumatic actuator or the electropneumatic actuators may, for example, be operated in the humidification mode so as to achieve a constant profile of the flow rate and/or the pressure.

For example, when resuming a high-flow therapy, a reduced respiratory air flow may initially be provided at the respiratory air outlet. For this purpose, a corresponding fan may be started up again particularly slowly so that the resulting respiratory air flow remains for a suitable period of time less than is actually intended for the high-flow therapy. The effect of the respiratory air being mixed with the ambient air at the respective patient interface in this case is that the inhaled air mixture does not exceed a critical temperature.

According to one embodiment, the operation of the respiratory air humidifier in the preparatory mode may comprise: generating a control signal for controlling at least one electropneumatic actuator so that a flow rate of the respiratory air provided at the respiratory air outlet at the end of the operation of the respiratory air humidifier in the preparatory mode reaches a final value that is equal to or less than a flow rate setpoint value which the flow rate is intended to have at the start of a subsequent operation of the respiratory air humidifier in the humidification mode. For example, the flow rate in the preparatory mode may be increased gradually to the flow rate setpoint value or to a value slightly (for example at most about 10% or at most about 5%) less than the flow rate setpoint value. It is therefore possible to avoid fluctuations of the flow rate, which may possibly feel unpleasant, when switching from the preparatory mode to the humidification mode.

According to one embodiment, the operation of the respiratory air humidifier in the preparatory mode may comprise: generating a control signal for controlling at least one electropneumatic actuator so that a pressure of the respiratory air provided at the respiratory air outlet at the end of the operation of the respiratory air humidifier in the preparatory mode reaches a final value that is equal to or less than a pressure setpoint value which the pressure is intended to have at the start of a subsequent operation of the respiratory air humidifier in the humidification mode. For example, the pressure in the preparatory mode may be increased gradually to the pressure setpoint value or to a value slightly (for example at most about 10% or at most about 5%) less than the pressure setpoint value. It is therefore possible to avoid fluctuations of the pressure, which may possibly feel unpleasant, when switching from the preparatory mode to the humidification mode.

According to one embodiment, the respiratory air humidifier may further comprise a ventilation hose, which can be heated by means of an electrical hose heater, for attachment to the respiratory air outlet. The ventilation hose may be attached to the respiratory air outlet when the respiratory air humidifier is in the operationally ready state. The respiratory air humidifier may further be formed in order to thermally regulate the respiratory air flowing through the ventilation hose additionally with the hose heater in the humidification mode. For example, it is therefore possible to prevent the respiratory air from being cooled too greatly on its way in the ventilation hose from the respiratory air outlet to the respiratory system of a patient who is being ventilated. In this case, the method may further comprise: preventing activation of the hose heater so long as the respiratory air humidifier is being operated in the preparatory mode. In this way, undesired additional heating of the respiratory air which flows through the ventilation hose in the preparatory mode, and possibly is already unpleasantly humid and/or unpleasantly warm, may be avoided.

According to one embodiment, a warning to warn a user of the respiratory air humidifier may further be generated when the respiratory air humidifier is switched to the preparatory mode. The warning may, for example, be an optical and/or acoustic and/or haptic indication. The warning may be given via the respiratory air humidifier itself and/or via a peripheral device, which is connected to the respiratory air humidifier wirelessly and/or by wire for data communication (e.g., via the Internet). Such a peripheral device may for example be a medical device (in particular a ventilator), a smartphone, a smartwatch, a tablet, a laptop or a PC. For example, the user may be prompted with the aid of the warning to take off a mask or nasal cannula attached to the respiratory air outlet, so that contact of the user's respiratory system with the respiratory air that is possibly too warm and/or too humid is avoided.

According to one embodiment, activation of the heating element may be prevented so long as the respiratory air humidifier is being operated in the preparatory mode. For this purpose, the heating element may for example be isolated from a power supply by means of a separate switch. For example, accidental activation of the heating element during operation of the respiratory air humidifier in the preparatory mode may therefore be avoided.

According to one embodiment, the respiratory air humidifier may further comprise: a respiratory air channel for conveying the respiratory air between the respiratory air inlet and the respiratory air outlet; a container for storing a liquid to humidify the respiratory air, for example water or a liquid containing water. The heating element may in this case be formed in order to heat the liquid from the container. Accordingly, the respiratory air humidifier may be formed so that the respiratory air comes in contact with the liquid in the evaporated and/or heated state while flowing through the respiratory air channel. The respiratory air may thereby be humidified before it emerges at the respiratory air outlet.

In addition, the respiratory air humidifier may comprise one or more electropneumatic actuators for controlling a respiratory air flow through the respiratory air channel.

### BRIEF DESCRIPTION OF THE DRAWINGS

Embodiments of the invention are described below with reference to the appended drawings. Neither the description nor the drawings are to be understood as a restriction of the scope of the invention. In the drawings:

FIG. 1 shows a respiratory air humidifier according to one embodiment of the invention.

FIG. 2 shows a flowchart of a method according to one embodiment of the invention.

The drawings are purely schematic and not to scale.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

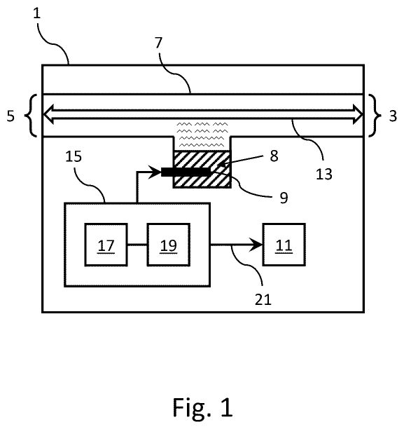

FIG. 1 shows a respiratory air humidifier 1 for use in combination with a ventilator. In this example, the respiratory air humidifier 1 comprises a respiratory air inlet 3 for attaching the ventilator, so that respiratory air from the ventilator can flow into the interior of the respiratory air humidifier 1, a respiratory air outlet 5, a respiratory air channel 7 for conveying the respiratory air between the respiratory air inlet 3 and the respiratory air outlet 5, a container 8 for storing a liquid to humidify the respiratory air (for example water or a liquid containing water) and an electrical heating element 9 for heating the liquid from the container 8. The respiratory air humidifier 1 is formed so that the respiratory air comes in contact with the liquid in the evaporated and heated state while flowing through the respiratory air channel 7 from the respiratory air inlet 3 to the respiratory air outlet 5, so that the respiratory air is humidified (while being thermally regulated) before it emerges at the respiratory air outlet 5. For example, the respiratory air may be brought to a particular comfort temperature, at which the respiratory air feels pleasantly warm when inhaling, during the humidification by correspondingly controlling the heating element 9. The respiratory air outlet 5 may be attached to a patient's respiratory system via one or more ventilation hoses and/or a suitable patient interface, for example a respiratory mask, a nasal cannula or tubing, so that the patient can be ventilated with the respiratory air from the respiratory air humidifier 1.

In addition, the respiratory air humidifier 1 may comprise at least one electropneumatic actuator 11 for controlling a respiratory air flow 13 through the respiratory air channel 7. The actuator 11 may, for example, be a fan and/or an electrically controllable valve. It is possible that the actuator 11 (or at least one of the actuators 11) is arranged in a housing of the respiratory air humidifier. The actuator 11 (or at least one of the actuators 11) may, however, also be arranged in a housing of the ventilator.

Further, the respiratory air humidifier 1 comprises a control unit 15 for controlling operation of the respiratory air humidifier 1. The control unit 15 may be embodied as a component of the respiratory air humidifier 1 and/or arranged in the housing of the respiratory air humidifier 1. The control unit 15 may, however, also be embodied as a component of the ventilator and/or arranged in the housing of the ventilator.

The control unit 15 may be configured to switch the respiratory air humidifier 1 between a humidification mode and a preparatory mode to prepare the respiratory air humidifier 1 for operation in the humidification mode. The respiratory air humidifier 1 may be formed in order, in the humidification mode, to humidify respiratory air which is supplied—here via the ventilator—at the respiratory air inlet 3, the respiratory air being thermally regulated by means of the heating element 9, and to provide it in the humidified state as a respiratory air flow 13 at the respiratory air outlet 5, for example by correspondingly controlling the actuator 11 (or the actuators 11).

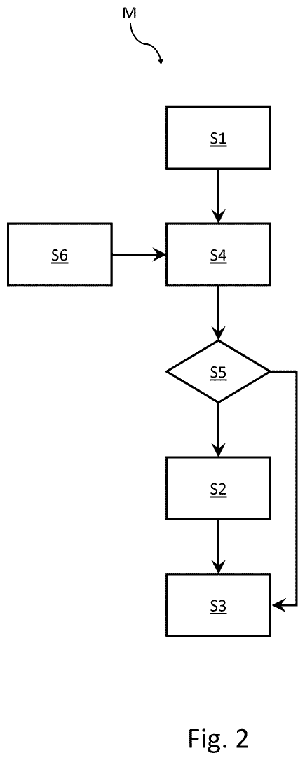

The control unit 15 may for example comprise a processor 17 and a memory 19, in which a computer program for operating the respiratory air humidifier 1 may be stored. The processor 17 may be configured to carry out the following method M (see FIG. 2) by running the computer program.

In a first step S1 switching of the respiratory air humidifier 1 on or off is detected, for example manual switching on or off, when for some reason the patient has interrupted or is about to interrupt the ventilation therapy for a short duration (for example for a duration of from about 30 seconds to about 3 minutes).

In response to the detection of switching on or off in step S1, the respiratory air humidifier 1 in a step S2 is switched to the preparatory mode and operated for a particular operating duration in the preparatory mode in order to actively and/or passively dissipate excess heat, which is due to operation of the respiratory air humidifier 1 in the humidification mode prior to switching on or off, from the interior of the respiratory air humidifier 1, for example from the respiratory air channel 7.

If switching of the respiratory air humidifier 1 on was detected in step S1, the (switched on) respiratory air humidifier 1 may for example in a step S3 be switched automatically to the humidification mode directly subsequent to the operation in the preparatory mode, and operated in the humidification mode until the respiratory air humidifier 1 is switched off again. It is also conceivable that after the end of the preparatory mode, the respective user is prompted by means of the respiratory air humidifier 1 and/or the ventilator to switch the respiratory air humidifier 1 to the humidification mode after the end of the preparatory mode, by generating a corresponding acoustic and/or optical and/or haptic operating instruction.

If switching of the respiratory air humidifier 1 off was detected in step S1, the (switched off) respiratory air humidifier 1 may continue to remain switched off subsequent to the operation in the preparatory mode, without a further response taking place. It is also possible that the respiratory air humidifier 1—even though it has been switched off—is still operated for a particular operating duration in the preparatory mode (immediately) after switching off, in order to enable sufficient cooling of the respiratory air humidifier 1 and/or to accelerate the cooling of the respiratory air humidifier 1, before the respiratory air humidifier 1 is (manually) switched on again, for example because the patient wishes to resume the ventilation therapy.

Expediently, the respiratory air humidifier 1 may comprise an additional battery for the power supply of the respiratory air humidifier 1 in the off state, for example during operation in the preparatory mode. The additional battery may, for example, be rechargeable. In this case, the respiratory air humidifier 1 may be formed in order to charge the additional battery in the on state, for example during operation in the preparatory mode and/or the humidification mode. In this way, it is possible to ensure that the battery is charged sufficiently each time the respiratory air humidifier 1 is switched off.

It is possible that the respiratory air humidifier 1 is (first) switched to the preparatory mode each time it is switched on or off. The respiratory air humidifier 1 may in this case be operated in the preparatory mode for example for a rigidly predefined operating duration, preferably from about 30 seconds to about 5 minutes.

Alternatively, the switching to the preparatory mode in step S2 may take place temperature-dependently. It is also possible that the duration of the operation of the respiratory air humidifier 1 in the preparatory mode is controlled temperature-dependently. For this purpose, in an optional step S4 as a response to the detection of switching on or off in step S1, a temperature value that indicates a current temperature of the respiratory air in the interior of the respiratory air humidifier 1, for example in the respiratory air channel 7, may be received in the control unit 15. The temperature value may be measured and/or estimated.

In a step S5 the temperature value may subsequently be compared with a particular threshold value. The threshold value indicates a temperature threshold at which the respiratory air just still feels comfortable when inhaling. The threshold value may, for example, be stored in the memory 19. The threshold value may be rigidly predefined or adjustable by the respective user via a suitable user interface.

If the comparison in step S5 reveals that the temperature value exceeds the threshold value, i.e. the current temperature lies above the temperature threshold, in step S2 the respiratory air humidifier 1 may be switched to the preparatory mode and operated in the preparatory mode.

If, on the other hand, the comparison in step S5 reveals that the temperature value does not exceed the threshold value, i.e. the current temperature is equal to the temperature threshold or lies below the temperature threshold, it is possible that switching to the preparatory mode is prevented. Instead, in step S3 the respiratory air humidifier 1 may for example be switched to the humidification mode (immediately) after switching on and operated in the humidification mode, preferably until the respiratory air humidifier 1 is switched off again.

It is possible that the temperature value is received in step S4 in a plurality of successive time intervals starting from the detection of switching on or off in step S1. The temperature value of each time interval may in this case be compared with the threshold value in step S5. This enables temperature-dependent control of the duration of the operation in the preparatory mode, by the operation in the preparatory mode being ended automatically as soon as the temperature value no longer exceeds the threshold value. For example, the (switched on) respiratory air humidifier 1 may be switched automatically from the preparatory mode to the humidification mode as soon as the temperature value no longer exceeds the threshold value.

The temperature value may preferably be a simple estimated value. This has the advantage that a separate temperature sensor for recording the current temperature in the interior of the respiratory air humidifier 1, for example in the respiratory air channel 7, may be obviated, which can reduce the production costs.

For example, the temperature value may be calculated in an additional step S6 by using a suitable mathematical model which approximates particular physical properties of the respiratory air humidifier. For this purpose, at least one measured and/or estimated value may be received in the control unit 15 and entered into the mathematical model. The mathematical model may be configured to convert the at least one input value into a corresponding temperature value and to output the temperature value as an output value.

The output value may then be further processed suitably in the control unit 15.

The input value may, for example, define at least one of the following variables:
a current heating power of the heating element 9;
a current filling level of the container 8;
a (for example average) duration of at least one heating phase, in which the heating element 9 was activated, prior to switching on or off;
a (for example average) duration of at least one cooling phase, in which the heating element 9 was deactivated, prior to switching on or off;
a current ambient temperature in the vicinity of the respiratory air humidifier 1;
a current relative air humidity expressed in terms of a current ambient temperature in the vicinity of the respiratory air humidifier 1;
a current ambient pressure in the vicinity of the respiratory air humidifier 1.

It is possible that a plurality of input values for at least two different variables, for example for at least two of the variables listed, are entered simultaneously into the mathematical model in order to calculate the temperature value.

The mathematical model may, for example, be stored in the form of a mathematical function and/or a lookup table in the memory 19. The parameters of the mathematical model may, for example, have been ascertained in tests.

The operation of the respiratory air humidifier 1 in the preparatory mode may also be restricted merely to preventing the respiratory air humidifier 1 from being switched to the humidification mode and/or to preventing the heating element 9 from being activated. This enables controlled passive thermal dissipation.

In addition or alternatively, controlled active thermal dissipation is possible. For this purpose, for example, the control unit 15 may generate one or more control signals 21 for controlling the actuator 11 (or the actuators 11) during operation of the respiratory air humidifier 1 in the preparatory mode.

By means of the control signal 21 (or the control signals 21), the actuator 11 (or the actuators 11) may for example be controlled so that:
a respiratory air flow 13 for dissipating the excess heat is generated through the respiratory air inlet 3 and/or through the respiratory air outlet 5 and/or through at least one vent opening of the respiratory air humidifier 1, which is separate from the respiratory air inlet 3 and from the respiratory air outlet 5;
the respiratory air is prevented from being provided at the respiratory air outlet 5;
the respiratory air is provided at the respiratory air outlet 5 with a lower pressure and/or a lower flow rate and/or a different flow direction than during operation of the respiratory air humidifier 1 in the humidification mode.
a flow rate of the respiratory air provided at the respiratory air outlet 5 follows an at least partially rising flow rate profile;
a pressure of the respiratory air provided at the respiratory air outlet 5 follows an at least partially rising pressure profile;
a flow rate of the respiratory air provided at the respiratory air outlet 5 at the end of the operation of the respiratory air humidifier 1 in the preparatory mode reaches a final value that is equal to or only slightly (for example at most 10% or at most 5%) less than a flow rate setpoint value which the flow rate is intended to have at the start of a subsequent operation of the respiratory air humidifier 1 in the humidification mode;
a pressure of the respiratory air provided at the respiratory air outlet 5 at the end of the operation of the respiratory air humidifier 1 in the preparatory mode reaches a final value that is equal to or only slightly (for example at most 10% or at most 5%) less than a pressure setpoint value which the pressure is intended to have at the start of a subsequent operation of the respiratory air humidifier 1 in the humidification mode.

The control options given in the list above may be implemented individually or in combination; at least two of the control options listed may be combined with one another. In addition, in step S2 a suitable warning to warn the respective user may be generated, for example before the respiratory air possibly feels too humid and/or too warm when inhaling. The warning may also prompt the respective user to perform a particular action, for example to wait for a short time and/or take off the respiratory mask or nasal cannula until the respiratory air has cooled sufficiently.

The ventilation hose attached to the respiratory air outlet 5 (or the ventilation hoses attached to the respiratory air outlet 5) may optionally be purposely heatable by means of an electrical hose heater. In this case, the respiratory air humidifier 1, for example the control unit 15, may be formed in order to thermally regulate the respiratory air flowing through the ventilation hose (or the ventilation hoses) additionally in the humidification mode by controlling the hose heater. Accordingly, the method M may comprise an additional step in which activation of the hose heater is prevented so long as the respiratory air humidifier 1 is being operated in the preparatory mode. In this way, undesired additional heating of the respiratory air which flows through the ventilation hose (or the ventilation hoses) in the preparatory mode, and possibly is already too humid and/or too warm, may be avoided.

Finally, it should be pointed out that terms such as “have”, “comprise”, “include”, “with”, etc. do not exclude other elements or steps and that indefinite articles such as “a” or “an” do not exclude the plural.

Further, it should be pointed out that features or steps that are described with reference to one of the embodiments above may also be used in combination with features or steps that are described with reference to other embodiments among the embodiments above.

### LIST OF REFERENCE NUMERALS

1 respiratory air humidifier
3 respiratory air inlet
5 respiratory air outlet
7 respiratory air channel
8 container
9 electrical heating element
11 electropneumatic actuator
13 respiratory air flow
15 control unit
17 processor
19 memory
21 control signal
M method
S1 detect switching on or off
S2 switch to preparatory mode
S3 switch to humidification mode
S4 receive temperature value
S5 compare temperature value
S6 calculate temperature value

What is claimed is:

1. A method (M) for operating a respiratory air humidifier, wherein the respiratory air humidifier comprises a respiratory air inlet, a respiratory air outlet and an electrical heating element, wherein the respiratory air humidifier can be switched between a humidification mode and a preparatory mode to prepare the respiratory air humidifier for operation in the humidification mode and is formed in order, in the humidification mode, to humidify respiratory air which is supplied at the respiratory air inlet, the respiratory air being thermally regulated by the heating element, and to provide it in the thereby humidified state at the respiratory air outlet, wherein the method (M) comprises:
when the respiratory air humidifier is switched on or off: switching (S2) the respiratory air humidifier to the preparatory mode and operating the respiratory air humidifier in the preparatory mode in order to enable dissipation of excess heat, which is due to operation of the respiratory air humidifier in the humidification mode prior to switching on or off, from the interior of the respiratory air humidifier, the heating element being deactivated.

2. The method (M) of claim 1, wherein the method further comprises, when the respiratory air humidifier is switched on:
switching (S3) the respiratory air humidifier to the humidification mode subsequent to the operation of the respiratory air humidifier in the preparatory mode, and operating the respiratory air humidifier in the humidification mode.

3. The method (M) of claim 1, wherein the method further comprises, when the respiratory air humidifier is switched on or off:
receiving (S4) a temperature value which indicates a current temperature of respiratory air in an interior of the respiratory air humidifier;
comparing (S5) the temperature value with a threshold value, which indicates a temperature threshold at which the respiratory air still feels comfortable when inhaling;
if the temperature value exceeds the threshold value: switching (S2) the respiratory air humidifier to the preparatory mode and operating the respiratory air humidifier in the preparatory mode.

4. The method (M) of claim 3, wherein the method further comprises, when the respiratory air humidifier is switched on:
if the temperature value does not exceed the threshold value: switching (S3) the respiratory air humidifier to the humidification mode and operating the respiratory air humidifier in the humidification mode.

5. The method (M) of claim 3,
wherein the temperature value is received in a plurality of successive time intervals and is compared with the threshold value in each time interval, the respiratory air humidifier being operated in the preparatory mode until the temperature value no longer exceeds the threshold value in one of the time intervals.

6. The method (M) of claim 3, wherein the method further comprises:
calculating (S6) the temperature by using a mathematical model of the respiratory air humidifier, at least one input value being received and entered into the mathematical model, the mathematical model converting the at least one input value into the temperature value and outputting the temperature value as an output value, the at least one input value indicating at least one of the following variables:
a heating power of the heating element;
a filling level of a container for storing a liquid to humidify the respiratory air;
a duration of at least one heating phase, in which the heating element was activated, prior to switching on or off;
a duration of at least one cooling phase, in which the heating element was deactivated, prior to switching on or off;
an ambient temperature in a vicinity of the respiratory air humidifier;
a relative air humidity expressed in terms of an ambient temperature in a vicinity of the respiratory air humidifier;
an ambient pressure in a vicinity of the respiratory air humidifier.

7. The method (M) of claim 1,
wherein operation of the respiratory air humidifier in the preparatory mode comprises:
generating a control signal for controlling at least one electropneumatic actuator in order to generate an air flow for dissipating excess heat through the respiratory air inlet and/or through the respiratory air outlet and/or through at least one vent opening of the respiratory air humidifier, which is separate from the respiratory air inlet and from the respiratory air outlet.

8. The method (M) of claim 1,
wherein operation of the respiratory air humidifier in the preparatory mode comprises:
generating a control signal for controlling at least one electropneumatic actuator in order to prevent the respiratory air from being provided at the respiratory air outlet; and/or
generating a control signal for controlling at least one electropneumatic actuator in order to provide respiratory air at the respiratory air outlet with a lower pressure and/or a lower flow rate and/or a different flow direction than during operation of the respiratory air humidifier in the humidification mode.

9. The method (M) of claim 1,
wherein operation of the respiratory air humidifier in the preparatory mode comprises:
generating a control signal for controlling at least one electropneumatic actuator so that a flow rate of respiratory air provided at the respiratory air outlet follows an at least partially rising flow rate profile and/or a pressure of respiratory air provided at the respiratory air outlet follows an at least partially rising pressure profile.

10. The method (M) of claim 1,
wherein operation of the respiratory air humidifier in the preparatory mode comprises:
generating a control signal for controlling at least one electropneumatic actuator so that a flow rate of respiratory air provided at the respiratory air outlet at an end of the operation of the respiratory air humidifier in the preparatory mode reaches a final value that is equal to or less than a flow rate setpoint value which the flow rate is intended to have at a start of a subsequent operation of the respiratory air humidifier in the humidification mode; and/or
generating a control signal for controlling at least one electropneumatic actuator so that a pressure of respiratory air provided at the respiratory air outlet at an end of the operation of the respiratory air humidifier in the preparatory mode reaches a final value that is equal to or less than a pressure setpoint value which the pressure is intended to have at a start of a subsequent operation of the respiratory air humidifier in the humidification mode.

11. The method (M) of claim 1,
wherein the respiratory air humidifier further comprises a ventilation hose, which can be heated by an electrical hose heater, for attachment to the respiratory air outlet;
wherein the method (M) further comprises: preventing activation of the hose heater so long as the respiratory air humidifier is being operated in the preparatory mode.

12. A control unit, wherein the control unit is configured to carry out the method (M) of claim 1.

13. A respiratory air humidifier, wherein the humidifier comprises a respiratory air inlet, a respiratory air outlet, an electrical heating element and the control unit of claim 12, wherein the respiratory air humidifier can be switched between a humidification mode and a preparatory mode to prepare the respiratory air humidifier for operation in the humidification mode and is formed in order, in the humidification mode, to humidify respiratory air which is supplied at the respiratory air inlet, the respiratory air being thermally regulated by the heating element, and to provide it in a thereby humidified state at the respiratory air outlet.

14. A computer program, wherein the program comprises instructions which cause the control unit of claim 12 to carry out a method (M) when the computer program is run by the control unit, the method comprising, when the respiratory air humidifier is switched on or off: switching (S2) the respiratory air humidifier to the preparatory mode and operating the respiratory air humidifier in the preparatory mode in order to enable dissipation of excess heat, which is due to operation of the respiratory air humidifier in the humidification mode prior to switching on or off, from the interior of the respiratory air humidifier, the heating element being deactivated.

15. A computer-readable medium, on which the computer program of claim 14 is stored.
